(19) United States
(12) Patent Application Publication
(10) Pub. No.: US 2014/0273052 A1
(43) Pub. Date: Sep. 18, 2014

(54) CHEMICAL SENSING APPARATUS HAVING MULTIPLE IMMOBILIZED REAGENTS

(71) Applicants: Rakesh Reddy, Deerfield Beach, FL (US); Bruce Johnson, Deerfield Beach, FL (US); Kevin Doyle, Deerfield, FL (US)

(72) Inventors: Rakesh Reddy, Deerfield Beach, FL (US); Bruce Johnson, Deerfield Beach, FL (US); Kevin Doyle, Deerfield, FL (US)

(21) Appl. No.: 13/844,665

(22) Filed: Mar. 15, 2013

(51) Int. Cl. G01N 33/18; C12Q 1/04; G01N 21/77

(52) U.S. Cl. CPC G01N 33/1893; G01N 21/77; C12Q 1/04
USPC 435/25; 435/288.7; 436/164; 422/82.09; 422/82; 435/34

(57) ABSTRACT

A sensor system in a water treatment system has a housing, controller, one or more light sources, one or more sensors and one or more targets having an immobilized reagent thereon. Light source emits light energy into the housing that is incident upon the target with the immobilized reagent and the reagent being in contact with water from the system. The immobilized reagent interacts with a reactant in the water such that the interaction changes the state of the reagent. When energy from the light source is incident on the target with the immobilized reagent the energy shows a change detectable by the sensor such that the changed energy is detectable by and collected at the sensor and data on the energy is communicated to the controller. The data is then correlated as a representation of a desired variable to be measured for the water in the water treatment system.

### COPYRIGHT NOTICE

Portions of the disclosure of this patent document contain material that is subject to copyright protection. The copyright owner has no objection to the facsimile reproduction by anyone of the patent document or the patent disclosure, as it appears in the U.S. Patent and Trademark Office patent file or records, but otherwise reserves all copyright rights whatsoever.

### BACKGROUND OF THE INVENTION

1. Field of the Invention

The invention relates to an apparatus for sensing variables in a body of water, more specifically to a system for sensing with an immobilized reagent in a surface of a material with a matrix a reactant/component in the water of a water treatment system and sensing a change in the surface indicating a measurement of a concentration of the particular variable.

2. Background of the Invention

Water treatment systems require consistent monitoring of various chemical and physical properties to maintain or adjust the system for a desired result. This is true of systems for recreational uses, such as but certainly not limited to pools, spas, water features, fountains, public water displays, public recreational water parks, and the like. As well as for instance in systems for industrial uses, such as but certainly not limited to boilers, HVAC, water conditioning systems, industrial processing systems, food processing, industrial cleaning systems, potable water systems, waste water systems, environmental monitoring systems, agricultural systems, aquaculture systems, testing labs, hydroponic farms, fish farms, bio-fuels industry, or other uses for one of the most plentiful resources on the planet. As such, a key element in any water management system having a flow of is the ability to accurately, efficiently, and repeatedly sense the condition of the water.

Several sensor methods and devices are available to measure these conditions. Amongst the many techniques for sensing, the two most popular techniques can be classified generally as those that use an electrochemical reaction and those that use a chemical detector serving as an indicator. Though more exotic forms of sensing exist these two classes represent the majority of the known and commercially available sensors in recreational and industrial uses today.

One such electrochemical method is used typically as a measurement of pH via a concentration of hydronium ions; the electrochemical properties of the analyte may be used to produce an electrical signal at a specially designed probe. These indicators use an ion selective electrode that acts as a transducer to convert the activity of a specific ion dissolved in a solution into an electrical signal that is then measured by the circuitry associated with the sensors. The sensing part of the electrode is made of an ion specific membrane. The principal drawback is that only some elements can be sensed in this fashion, for instance ions of hydrogen, sodium, silver, lead and cadmium and related molecules that ionize are subject to this type of sensing. They are not capable of effectively sensing concentrations of analytes that do not ionize.

Additionally, because the ion-exchange is conducted through a membrane, typically glass, it is possible that some other ions will interact and distort the value of the target ions. This also creates a need for special handling and storage between measurements, as the membrane should be kept in the solution to prevent the membrane from drying out. Also, in the case of pH reporting, when dealing with solutions having low concentrations of detectable ions, an interfering alkali metal can cause the pH reporting to be non-linear. Similarly with solutions with high hydrogen ion concentrations the influence of anions in the solutions makes the sensor output nonlinear.

Some attempts have been made to provide liquid and gel filled electrodes to obviate some of these issues. These liquid and gel filled electrodes tend to bleed or leak their solutions out over time. This means that electrodes have a lower service lifetime in an in-situ testing environment. Additionally, both in the case of dry and liquid/gel filled electrodes, the manufacturing of electrodes is a complex process making them expensive to produce. These types of sensors are difficult to manufacture, difficult to deploy in a number of sensing environments, require constant maintenance due to their fragility and fail to provide sufficient accuracy over long term measurement of target environments, so much so that even static electricity can interfere causing erratic readings from these types of sensors.

Similarly, in instances where there are no electrode sensors available to measure a particular variable, such as in situations measuring free chlorine concentrations Cl⁻), which is particularly relevant in water purification systems and recreational applications, electrolytic systems and sensors have been developed to measure oxidation reduction potential (ORP) of a solution. The ORP of a solution is the tendency of a chemical species to give or acquire electrons and thereby be oxidized or reduced respectively. How easily a substance can get oxidized/reduced in a solution is given by a potential that is referenced to the redox potential of hydrogen ion/hydrogen which is assigned a standard potential of 0 millivolts. So in these testing schemes, a measured ORP value is associated with a certain concentration of an analyte.

The ORP system has two electrodes, one that has an inert metal electrode that will give up electrons to an oxidant or accept electrons from a reductant, typically done through electrolysis at a blade or similar structure. The system needs a reference electrode to complete the circuit. This gain or loss causes a change in voltage that is used to calculate ORP and relate the concentration of chlorine. The problems with this technique are many. The ORP value depends on the concentration of all the ions in the system. As a result ORP is not directly calculating the value of the target analyte, for instance chlorine, but is indirectly estimating it rendering these systems less accurate. The ORP also depends on the pH of the sample in the circuit. Systems account for this my taking into consideration the pH of the system, however any error in this pH reading will translate to variations in the ORP as well resulting in overall inaccuracies in the sensing in the system. The electrodes sets are also expensive both in their construction and due to the special handling required to maintain them without fouling or damaging them. Additionally, the voltages produced are small requiring complex electronics and wiring to operate, again increasing costs and complexity.

Finally, the most debilitating shortfall of these devices is that they require constant calibration on site. Over time the electrolysis process fouls the electrodes with build up from the separation of the molecules on the blades. Over long term use, the electrolysis reaction also erodes the electrodes. Both of these occurrences creates drift in the measurements made by these sensors which forces recalibration to maintain accuracy and, in the case of corrosion, shortens operational life, which increases operational costs.

Thus there are several problems with electrode sensor processing. The most widely deployed sensor systems with such sensor technologies suffer most greatly from drift that occurs due to the interaction of analytes and sensor components and buildup on the sensors components. For this reason, the electrodes do not give a reproducible electromotive force over long periods of time. This requirement for frequent calibration relegates this type of measurement to laboratory environments ideally. However, several companies attempt to utilize this technology to sense pH or chlorine in things like water displays, pools, and spas. This results in an increased need for maintenance and a very costly sensor setup, as not only is there significant issues with fouling, but the increased cost of maintaining and/or replacing the probes as well as the errors and unnecessary treatment or lack of treatment as the case may be of the water system. Some examples of these types of device can be seen in the HM Digital TDS-EZ Water Quality TDS Tester, European patent EP1847513B1 to Gaspar, and US Patent Application US 20120234696 A1 all of which are examples of attempted electrode solutions.

The second sensor type typically used is based on chemical reactions in a mixture. The ubiquitous method of testing concentration of reactants/analytes in a solution is through observation of the effect that the reactant has on a reagent that is specifically designed to interact with the reactant. This type of testing can range from reagents that change their absorption properties to those that create detectable precipitates and vary in delivery method from liquids, ranging from sprays to liquids in handheld devices, to paper impregnated strips in testers and test kits.

Reagents that exhibit chromism, e.g. change color, can exhibit this in different ways, including for example, but certainly not limited to absorption of incident or reflected light, absorption of energy and readmission in the same spectrum, absorption of light followed by the emission of light in a different spectrum, the change in the polarization characteristics of the light or the like. However, one of the many drawbacks of such detection methods is the addition of a reagent to a sample to instigate a color changes can result in uncontrolled absorption of the reagent by the water making re-sampling a necessity and potentially skewing any measurements.

These systems excel, therefor, in detecting one off reactions without regard for absorption or fouling of the sample with the reagent, for instance in a laboratory where mixing by hand in test tubes is sufficient and visual detection of changes is sufficient or during the operation of a pool by a visual detection test by a lifeguard. However, in preparing large numbers of samples or where higher accuracy measurements against the sample are needed or where consistent, automated, high repetition, real time sampling is needed, these systems fall short.

One example of a impregnated strip based system is the Aqua Check TRUTEST® Digital Test Strip Reader. The strip is dipped in a sample and analyzed by the handheld unit. The strip system relies on restocking of the strips. Similarly, some companies have provided for automatic visualization of a color changing reagent with measurement. For example, the Palintest Chlorometer and Palintest Photometer 7500 which are examples of systems that use chromism of a reagent and measures same on the lab bench or in the field, respectively. These systems are still limited to a single samples with a separate liquid reagent that must be replaced in controlled manner into each sample and must be calibrated.

There are also systems that allow multiple reagents to be added at one time to a single body of water and they automate the measurements across all the reagents. A still further version of automation in sensing in a color changing sensor system, as seen in the Lamotte WATERLINK SPIN LAB, which provides for a version of a system using multiple chromism reagents and a single sample to determine multiple variables at once. However, each sample expends a disk and the disks with the reagents must be restocked, recalibrated, and the sample must be brought manually to the tester.

Finally there are systems that use color changing reagent-reactant reactions and do automatic reagent dispensing in an automatic chemical analyzer. These are systems that have storage containers for reagents and then pump them into a testing chamber. U.S. Pat. No. 4,070,156 shows a system having such reagents being automatically pumped to test chambers with a sample to cause a reagent-reactant reaction in a test chamber. This system is still very complex and very expensive. The system requires liquid reagents be combined with each sample and still requires restocking of the reagents used in the system as well as recalibration of the system as between samples.

Improvements and developments in materials science and technology have allowed for the development of a new class of sensors and sensor technologies. Based on the Sol-Gel process, the development of rigid surfaces having entrapped and immobilized reagents has been made possible. A typical method of manufacture is described hereafter. Although a description of a form of Sol-Gel production is provided herein, the example is meant to be non-limiting. Other forms and formats for creating Sol-Gel materials can be used without departing from the spirit of the invention with a goal of providing a porous structure with embedded, immobilized reagents. In a principal step of a typical manufacturing process of a Sol-Gel material, hydrolysis of the colloidal components is conducted. This is where a precursor such as Tetraethoxy silane (TEOS), Tetramethoxy silane (TMOS), Methyltrimethoxy silane (MTMOS) or other metal alkoxides are hydrolyzed. The hydrolysis requires a catalyst typically a very small amount of water or acid or the like. Since these metal alkoxides are not immiscible in water, a solvent such as a base alcohol suitable for the metal alkoxide, for instance in the case of TEOS an ethanol, can also be used for phase transitions.

Following hydrolysis, the condensation of the material occurs where the individual precursor molecules start connecting to each other. The material then begins gelation where the system forms a viscous liquid. This is the step where the reagents will typically be added. Further cross-links on the molecular level are formed within the viscous liquid through a process called ageing. Ageing can also be accompanied by mechanical manipulation of the product. For instance, one example of forming a thin film can be, but is certainly not limited to, spin coating which is used to form a thin layer of xerogel. The xerogel is then dried such that alcohol/water in the solution is lost and all structural bond formations are completed. Post processing from a bulk material with the matrix structure and immobilized reagent can also occur to produce the sensor material. Finally, a process called densification can be used to thermally treat and collapse the open structures to form a dense ceramic.

The Sol-Gel process can produce a number of structures including for instance, but certainly not limited to thin films, bulk materials and dense ceramics based on the treatment and post treatment of the Sol-Gel. An example of a Sol-Gel substrate can be found in U.S. Patent Application No. 2008/0311390 to Seal, et al. The Sol-Gel process allows for creation of a matrix structure and the doping of the structure with reagents which are encapsulated or entrapped and immobilized within the matrix. The immobilization may be assisted with additional chemicals which affect forces for affixing the reagents within the matrices of the surface or material. This allows for a detectable change on the Sol-Gel target pad that results from the reaction of the immobilized reagent with reactants in solution, whereby the immobilized reagent remains in the material and therefore obviates a need for restocking of the reagent. The result is a surface that will react optically with a sample.

Some devices using this material sensors have been developed for laboratory sensing. One example of such a device from Oceanoptics is a Sol-Gel matrix material with a reagent such as bromocresol green coated on cuvettes. However, to date no such devices have been developed for use as a system in a body of water or water treatment system, much less one which is in-situ and/or retrofittable to an existing water treatment system with the ability to sample and monitor one or more parameters using a target having an immobilized reagent target and sensor.

A still further aspect of the invention is to provide an in-situ sensor system with an automatic recalibration capability and an at least one immobilized reagent.

There exists a need for a system utilizing new materials technologies to develop an in-situ sensor system for use in a water treatment system that is more accurate, more cost effective, requires less calibration, requires no restocking of reagents, provides more repeatable results, has a longer operational life and is easier to install and maintain than existing chemical sensors for use in water and water treatment systems that does not use liquid reagents that need to be replenished, does not require complex pumps, does require expensive probes, does not suffer from drift and does not present issues with respect to special storage or handling requirements.

A need exists for a both an original equipment and retrofittable versions of the sensor system with immobilized reagents thereon that can be incorporated into any water treatment system so as to be put in contact with the flow of water in a water treatment system and produce a measurable reaction that can be correlated with measurements of desired parameters of the water in the water treatment system. A need also exists for a controller for controlling the analysis of the reaction at the sensor.

### SUMMARY OF THE INVENTION

An aspect of the invention is to provide an in-situ sensor system for use in a water treatment system that is more accurate, more cost effective, can sense more analytes, is more selective in sensing of analytes provides more repeatable results, has a longer operational life and is easier to install and maintain than existing chemical sensors for use in water treatment systems.

Another aspect of the invention is to provide an original equipment and a retrofittable versions of a sensor system with immobilized reagents thereon that can be incorporated into any water treatment system so as to be put in contact with the flow of water in a water treatment system and produce a measurable change that can be correlated with measurements of desired parameters of the water in the water treatment system.

A still further aspect of the invention is provision of a sensor system for a water treatment system that utilizes at least one Sol-Gel thin film target surface with an immobilized reagent therein for spectrographic and/or optical interactions in a sample of the water with at least one immobilized reagent resulting in sensor detectable data and analyzing this data to compute a representation of a desired value for one or more variables of the water.

A still further aspect of the invention is provision of a sensor system for a water treatment system that utilizes at least one Sol-Gel thin film target surface with an immobilized reagent therein for spectrographic manipulation of reactants in a sample of the water with multiple immobilized reagents resulting in sensor detectable data and an at least one calibrating surface or blank for automatic and instant recalibration of the device.

Yet another aspect of the invention is improved techniques for bringing a test solution to an immobilized reagents in a target surface.

Still further aspects are directed to improved techniques that are used to create cost effective sensing techniques that sense wavelength specific changes in the absorption and/or fluorescence and/or luminescence profiles of a reagent.

An aspect of the invention is the provision of a method and computer software on a computer that provides a processes for calibration of the sensor having an immobilized reagent thereon.

A further aspect of the invention is to provide a sensor system that is not selectively skewed based on an ion concentration level or other aspect of the sample.

The invention includes a method, an apparatus, and an article of manufacture for sensing a parameter of a flow of water in a water treatment system.

The apparatus of the invention includes a sensor system in a water treatment system, having a housing, a controller, an at least one light source, an at least one sensor. The sensory system further includes an at least one target having an at least one immobilized reagent with the at least one light source emitting light energy into the housing that is incident upon the at least one target with the immobilized reagent and the immobilized reagent being in contact with a sample of water from the water treatment system. The at least one target having the immobilized reagent interacts with a reactant in the water such that the interaction changes the state of the reagent and when energy from the at least one light source is incident on the at least one target with the immobilized reagent the energy from the at least one target having the at least one immobilized reagent shows a change detectable by the at least one detector such that the changed energy is detectable by and collected at the sensor and data on the energy is communicated to the controller, the data is then correlated as a representation of a desired variable to be measured for the water in the water treatment system. The optical profiles detected by the sensors can be stored on the controller along with calibration profiles as historical data.

The at least one target further comprises multiple targets with immobilized reagents. The sensor system having an at least one target with an immobilized reagent can include multiple immobilized reagents embedded in the at least one target. The multiple reagents can be on multiple targets The immobilized reagent can be at least one of an at least one organic or inorganic dyes. The at least one organic or inorganic dye can be at least one of bromocresol green, cresol red, bromothymol blue, bromopyrogallol red, phenol red, orthotolidine, N-N, diphenyl-p-phenylenediamine, and melamine. The immobilized reagent that is at least one of an at least one enzyme. The at least one enzyme can be at least one of Aequorin, Chloramine, and Glucose Oxidase. The variable can be measured by a concentration of the reactant and the reactant can be an at least one dissolved reactant in the water. The dissolved reactant can be an at least one ion. The at least one ion can be at least one of an at least one hydronium, chlorine, calcium, iron, sodium, lead bromine, magnesium, and copper ion The dissolved reactant can be an at least one compound. The at least one compound can be an at least one of an at least one oxygen, carbon-dioxide, cyanuric acid, chlorine, and glucose compound. The variable can be measured by a concentration of at least one of a flora and fauna. The at least one flora and fauna can be an at least one algae and bacteria.

The sensor system can further include a reflector portion or chamber. The sensor system can also include sensors for flow rate, temperature, and similar variables. The controller can be within the housing in an electronic section also housing the at least one light source and the at least one sensor. The sensor system can include an at least one window separating the at least one light source from a flow of water within the housing, wherein the targets can be spaced around the window and the sensors can be located proximate to the at least one target. The sensor system can further include a reflective portion of the housing whereby light emitted by the at least one light source and can be emitted through the window and can be reflected back within the reflective portion back toward the at least one target and passes through the target into a light chamber which aids in collecting and focusing the reflected light onto the at least one sensor above the target.

The at least one target with an immobilized reagent can be comprised of material formed by a Sol-Gel process. The matrix can be formed using a metal alkoxide or a metal alkyloxide precursor compound in the Sol-Gel process. The precursor compound can be one or more of Tetraethoxy silane (TEOS), Tetramethoxy silane (TMOS), and Methyltrimethoxy silane (MTMOS). The Sol-Gel formed material can be at least one of an at least one thin film, bulk material and dense ceramic.

The controller can be outside of the housing. The sensor system can include a user interface. The controller can be located with the user interface. The controller can be located on the housing and can include a communication subsystem for wired or wireless communication with a graphical user interface. The housing can be in line with a plumbed water line in the water treatment system. The at least one sensor can be at least one of an at least one photodetector. The at least one photodetector includes an at least one spectrometer, CMOS chip, CCD chip, photodiodes, photoresistors, phototransistors, and phototubes. The targets can be directly in the line of flow. The water flow can be redirected from the main line of water flow to the targets and then back to the main line of water flow. The housing can containing the target can be in the flow of water. The housing can contain the target can be in a body of water being serviced by the water treatment system.

The housing can be coupled via a pressure differential to divert a sample of the water in the water treatment system to the sensor system. The housing can be coupled to the a pipe in the water treatment system through an upper and lower collar portion with an inlet and an outlet path extending from the housing into the pipe to redirect water into the housing. The sensor system can further include an at least one additional sensor additionally measuring an at least one or more of temperature, humidity, ambient light conditions, free chlorine, flow displacement, and differential pressure.

The sensor system can also include an at least one calibration target or blank, where light incident on the at least one immobilized reagent target can be also incident upon the calibration target or blank without an interaction and variations in the profile of the energy emitted from the at least one light source can be detected by the at least one sensor, whereby any variations in the received profile can be used to adjust the sensors and correct the data for the light received that can be incident on the at least one immobilized reagent target. The variation in the profile of the energy emitted and received by the at least one sensor at the at least one calibration target or blank can be stored by the controller.

The stored data on variations in the profile of the energy emitted can be reviewed by the controller and the controller can categorize and thereby detect profiles for fouling of the water treatment system flow of water, errors from the at least one light source, errors from one or more of the at least one sensors, and the stored data can be compared against calibration data stored during manufacture of the sensor system. The data correlated as a representation of a desired variable to be measured for the water in the water treatment system can be communicated through a user interface. The user interface can be on the housing. The user interface can be wirelessly coupled to the controller. The user interface can be a mobile computing device. The user interface can be coupled via a wired coupling to a user interface outside the housing.

The apparatus of the invention includes an at least one sensor system coupled to a pool, spa, or water feature, the water treatment system having water flowing within the system, the sensor system having an at least one housing, an electronic section containing at least one light source, at least one controller, at least one sensor, an at least one immobilized reagent target. The sensory system has an at least one sensor sensing energy incident on or through the at least one immobilized reagent target, wherein the at least one light emits an energy with a specific known optical profile which is then incident on the at least one immobilized reagent target which is in contact with the water from the water treatment system and the immobilized reagent interacts with the water sample to produce a reaction in or on the at least one immobilized reagent target which changes the energy profile on the at least one immobilized reagent target, the changes are then detected by the at least one sensor sensing energy incident on or through the at least one immobilized reagent target.

The sensor system can include a calibration target or blank, where light incident on the at least one immobilized reagent target can be also incident upon the calibration target or blank without an interaction and variations in the profile of the energy emitted from the at least one light source, whereby any variations in the received profile can be used to adjust the sensors and correct the data for the light received that can be incident on the at least one immobilized reagent target.

The housing can be provided as a component of the existing water treatment system. The sensor system can be a component of a chlorine generator or water pump. The housing being placed in-line with a portion of the piping of the water treatment system. The sensor system can include a pipe portion having an inlet and an outlet with water flowing through the sensor system. The at least one light source can be at least one of an at least one ultraviolet, infrared, and visible light. The sensor system can further include an at least one top of the enclosure releasably retained to the housing and including a physical port and a power supply. The sensor system can further include an at least one display, user interface, and user input.

The display and user inputs can be digital and on a mobile device. The sensor system further includes analog tactile elements in a display and as user inputs. The display and user inputs can be digital and incorporated into a touch pad device. The display and user interface can be coupled wirelessly or coupled with a wired connection to the controller or to a master controller.

The sensor system can control one or more further components of the water treatment system. These further components can include an at least one of an at least one chlorinator, pump, heater, and pH dispenser. The housing can be remote from the water system and includes an at least one diverter to sample water from a portion of the water treatment system. The housing can have an at least one pump and the controller can administer water treatment system chemical solutions. The housing has an upper and lower collar portion and can be coupled through a pipe within the water system by the collar portions.

The sensor system can include an inlet portion and an outlet portion that protrude from the housing into the water flow within the pipe. The immobilized reagent that can be at least one of an at least one organic or inorganic dyes. The at least one organic or inorganic dye can be at least one of bromocresol green, cresol red, bromothymol blue, bromopyrogallol red, phenol red, orthotolidine, N-N, diphenyl-p-phenylenediamine, and melamine. The sensor system can further include an immobilized reagent that can be an at least one of an at least one enzyme. The at least one enzyme can be at least one of Aequorin, Chloramine, and Glucose Oxidase. The variable can be measured by a concentration of the reactant and the reactant can be an at least one dissolved reactant in the water. The dissolved reactant can be an at least one ionic compound. The at least one ion compound can be at least one of an at least one hydronium, chlorine, calcium, iron, sodium, lead bromine, magnesium, and copper ion. The dissolved reactant can be an at least one compound. The at least one compound can be an at least one of an at least one oxygen, carbon-dioxide, cyanuric acid, chlorine, and glucose compound. The variable can be measured by a concentration of at least one of a flora and fauna The at least one flora and fauna can be an at least one algae and bacteria.

The method of the invention includes a method of sensing a reactant in a body of water, comprising the steps of directing a sample of water from a body of water into a housing having an at least one detection targets with an immobilized reagent thereon; directing an at least one light source incident upon the at least one detection targets having immobilized reagents thereon; emitting energy from the at least one light source incident upon the at least one detection targets having immobilized reagents thereon such that the energy changes with any interaction the immobilized reagents have with the sample; detecting a change in the energy incident upon the at least one detection targets having immobilized reagents caused by the interaction of the immobilized reagents with the sample of water; and reporting the results of the detection step.

Moreover, the above objects and advantages of the invention are illustrative, and not exhaustive, of those which can be achieved by the invention. Thus, these and other objects and advantages of the invention will be apparent from the description herein, both as embodied herein and as modified in view of any variations which will be apparent to those skilled in the art.

### BRIEF DESCRIPTION OF THE DRAWINGS

Embodiments of the invention are explained in greater detail by way of the drawings, where the same reference numerals refer to the same features.

FIG. 1 shows a perspective view of an exemplary embodiment of the instant invention as deployed in a water treatment system.

FIG. 2 shows a cross-sectional view of the exemplary embodiment of FIG. 1.

FIG. 3 shows a further cross sectional view facing the top of the housing of the exemplary embodiment of FIG. 1.

FIG. 4 shows a plan view of an exemplary embodiment of the instant invention

FIG. 5A shows a flow chart for a method of calibrating an exemplary embodiment of the instant invention.

FIG. 5B shows a flow chart for a method of measurement of the instant invention.

FIG. 6 shows a perspective view of a further exemplary embodiment of the instant invention.

FIG. 7 shows a cross-sectional view of the embodiment of FIG. 6.

FIG. 8 shows a sensing component of a still further exemplary embodiment of the instant invention.

FIG. 9 shows an exemplary embodiment of a diversion component of the still further exemplary embodiment of FIG. 8.

FIG. 10 shows a plan view of an exemplary embodiment of the instant invention deployed in a water treatment system servicing a pool and/or spa.

FIG. 11 shows a plan view of an exemplary embodiment of the instant invention deployed in a water treatment system for a fish farm operation.

FIG. 12 is a chart showing a spectral profile of multiple light sources in an exemplary embodiment providing a controllable wavelength selection capability.

### DETAILED DESCRIPTION OF THE INVENTION

The instant invention is directed generally to a sensor system for a water treatment system having a housing that utilizes a sensor system which includes at its core a sensor target with an immobilized reagent entrapped in a molecular matrix which is capable of interacting with a reactant/analyte substance in concentration in the water of the water treatment system This entrapped reagent removes issues with corrosion, labor, maintenance, accuracy, repeatability, and lowers costs to sense parameters currently requiring a great deal of manual labor and attention. In addition, although the system can operate to sense a single parameter, the system can also be configured to sense multiple parameters. Thus, an exemplary embodiment includes elements capable of interacting and thereby sensing and reporting multiple variables for the water treatment system. The result would be a system capable of replacing a number of existing sensors, including for instance salinity, temperature, dissolved carbon di-oxide, calcium ions, potassium ions, ammonium compounds, pH, chlorine, oxygenation, and other aspects of the water in the water treatment system with a single sensing unit. This reduces overall costs and maintenance as well as the labor involved in determining these parameters. Finally, as evidenced by the various embodiments shown, a number of paths may be used in conjunction with the sensor system to fit and retrofit the sensors to existing components in a water treatment system.

FIG. 1 shows a perspective view of an exemplary embodiment of the instant invention as deployed in a water treatment system. An enclosure or housing 100 is provided as a component of a water treatment system, the housing 100 being placed in-line with a portion of the piping of the water treatment system. As shown in the FIG. 1 a pipe 105 is provided having an inlet 112 and an outlet 110, with water flowing through the system in the direction of the arrows shown. The inlet and outlet 112, 110 each couple to the plumbed water system using couplings 120 which thread onto pipe ends 125, as better seen in FIG. 2. In this exemplary embodiment the housing 100 incorporates an electronic section which contains at least one of an at least one sensor, at least one light source, at least one controller, and at least one immobilized reagent target sensor and similar additional electrical components.

Although referred to as a light source, the at least one light source may project visible light as just one non-limiting example. The term light source, however, includes any radiated energy source which when incident upon the immobilized reagent producing the reaction or interaction with the reactant in the sample that will have a measurable change in the energy and for which this change is detectable by the at least one associated detector or sensor. This can also include, but is certainly not limited to, ultra-violet, infra-red, and visible light as well as other types and frequencies of radiation.

A top of the enclosure 130 is provided and may be made removable to provide service access to the electrical section 115 in the housing 100. The exemplary embodiment shown provides screw threads (not shown) and a grip surface 135 to removably couple the enclosure top 130 to the enclosure, although any coupling member may be utilized that would provide for releasably retaining the top of the enclosure 130 to the housing 100. Additionally on the upper part of enclosure top 130 a physical port 137 is provided allowing access to wiring, including for example off-system communication leads, power sources, and the like. As seen in FIG. 1, a line runs into physical port 137 which includes a power supply 138 provided to power the electrical components in the electrical section 115 of the exemplary embodiment of FIG. 1.

On the upper portion 132 of the enclosure top 130, a display 150 is provided to show operational parameters of the instant invention and function with the user interface 140 in operating the exemplary embodiment. In addition the user interface 140 is provided having an at least one user input 145. In further embodiments the user interface can be coupled via a wired coupling to the controller or via a wireless coupling to the controller. In addition the controller may be enabled to communicate with a network, for instance a home Wi-Fi network, or through a wireless communications protocol, like Bluetooth, or through the internet or other network to a user interface 140. The user interface can also be for example, but certainly not limited to, on a mobile device or mobile computing device or tablet.

In the exemplary embodiment shown in FIG. 1, the user input 145 is shown as a set of buttons. However it would be understood to one of ordinary skill in the art, the user inputs could be replaced with analog tactile elements or could be incorporated as part of a touch pad or could be ported wirelessly or via a wired connection to a controller element or to a master controller away from the housing 100. The user input 145 may be of any type of input suitable for instructions from a user using the user interface and programming on the controller 165.

FIG. 2 shows a cross-section of exemplary embodiment of FIG. 1. The embodiment of FIG. 1 is placed in line in a plumbed water system similar to those further described in FIGS. 10 and 11 herein below. The sensing system 10 of the exemplary embodiment has housing 100 coupled via couplings 120 with the threaded portion of the couplings engaging threads 125 and securing the system at inlet 112 and outlet 110 with water flowing there between as indicated by the arrows.

Within the housing 100 a reflective section of pipe 155 is provided. The exemplary embodiment of FIG. 1 provides for a curved section of highly polished pipe that acts as a reflector of light within the housing 100. It should be noted that the reflector portion 155 of the housing 100 may take any necessary shape to achieve a necessary redirection of light energy from the at least one light source 175. This can include for example, but is certainly not limited to, curved, straight plane, or similar reflective structures. As shown here, one non-limiting example of this reflector section 155 can be a highly polished, curved portion of the housing that is integral to the housing 100. However, further methods and structures can be provided to construct the reflector portion or chamber 155. Other examples of such methods and structures include a separate element within the housing shaped from reflective material, coatings or other treatments to increase the reflectivity of a wall of the housing, or similar structures or methods to provide sufficient return of the energies being broadcast by the at least one light source 175. In addition to simply reflecting the energy of the at least one light source 175, the reflective section of pipe 155 may also be provided with filtering characteristics, such that it absorbs, modulates, fluoresces, or modifies the emanation of the energy from the at least one light source in a fashion for the sensor system 10. Some non-limiting examples of filters include dyes, die chromic, or other filtering mechanics. The filtering may be employed in any of the structures or coatings specified to provide the desired modification of energy being returned to the sensor system 10.

An electronics section 115 is provided within the housing 100 in an upper portion of the sensor system 10 and is sealed from water intrusion. Within the electronic section 115 an at least one light source 175 is provided. Although multiple sources are provided in the exemplary embodiment shown, a single source with sufficient luminescence would work equally well within the exemplary embodiment. In the exemplary embodiment shown, the at least one light source 175 is a series of LEDs which emit light toward the reflector section 155 passing the light through the window 180. Similarly window 180 may be comprised of for example, but certainly not limited to, glass, acrylic, poly carbonate, or similar material transparent to the spectrum of energy being emitted by the at least one light source 175. Again, as noted above with respect to the reflective section 155 a filter material may be placed at the lens 180 or on the surface of the at least one light source to modulate or otherwise effect the energy emissions of the at least one light. The window 180 may be comprised of a single portion or broken into several smaller windows. The window 180 may also contain filter material or be comprised of specially composed materials to form function of a filter for the light source or energy. This can include for example, but is certainly not limited to, thin-film coatings, color dyes, polarizations, or similar techniques to modify energy emitted by the at least one light source 175.

The at least one light source 175 can also include, but is certainly not limited to, ultraviolet, infrared, visible light as well as other types and frequencies of radiation including for example but certainly not limited to radio and microwave sources. A non-exhaustive list of examples of the sources include but are certainly not limited to incandescent lamps, fluorescent lamps, High Intensity Discharge lamps, Light Emitting Diodes, Laser Diodes and the like. It should also be noted that the at least one light source may include multiple independently controlled lights having different wavelength bands that are used selectively at one time or in combination with each other or a broad spectrum light source, as better seen in FIG. 4. These sources may be enabled independently so has to illuminate the immobilized reagent with specific wavelength at a given time so that the sensor can measure the change in the absorption, fluorescence, luminescence and similar characteristics between two states the different energy sources.

Additionally, as noted herein below, the required wavelengths that the reagent is illuminated with can also be provided via a set of filters such as but certainly not limited to absorption, interference, or other operational types of filter. It may be possible that the wavelength from a broadband source is selected using a tunable filter or similar selection mechanism. Additionally, the light may be polarized linearly or circularly and this can be achieved as a function of a primary light source or using a secondary filter/phase change mechanism.

An at least one target having an immobilized reagent 210, 240 entrapped thereon, for instance via a Sol-Gel production process, is depicted within the housing 100 in the exemplary embodiment as shown. These targets are shown as being in an annular ring just outside the window 180, as better seen in the bottom cross-section view of FIG. 3. The ring section or the entire electronic section can be made to be removable and replaceable either by the user or as part of a factory service program. The immobilized reagents can include, but are certainly not limited to organic or inorganic dyes such as but not limited to, bromocresol green, cresol red, bromothymol blue, bromopyrogallol red, phenol red, orthotolidine, N-N, diphenyl-p-phenylenediamine, melamine or enzymes such as, but certainly not limited to Aequorin, Chloramine, Glucose Oxidase and the like, used alone or in any functional combination with one another. The immobilized reagent-reactant activity is measured and is calculated to produce an output representing a variable. Variables can be for example, but are certainly not limited to, dissolved analytes that can be ions such as hydronium, chlorine, calcium, iron, sodium, lead bromine, magnesium, copper, and the like; or dissolved analytes that can be compounds such as oxygen, carbon-dioxide, cyanuric acid, chlorine, glucose and the like; or flora and fauna such as algae, bacteria, and the like, alone or together in any functional combination that may be measured. This occurs without the immediate loss or absorption of the immobilized reagents in the matrices of each of the targets 210-240. It should be noted that the target pads will retain sensitivity theoretically for a very long time as the immobilized reagents are contained in a structural and chemical bond to the surface. However, there may eventually be some degradation over a very long service life from a slight leaching losses in the immobilized reagent over the service life of the sensor pads. Above the at least one target is an at least one optical chamber 190 affecting the energy that passes through the target in the exemplary embodiment shown from the at least one light source 175 with at least one sensor 170 contained therein to detect the energy. The immobilized reagent of the target 210, 240 may have an initial absorption/fluorescence/luminescence state that is associated with the first state (such as concentration) of the analyte/reactant and a second different state that is associated with the second state (such as concentration) of the analyte/reactant. Although the exemplary embodiment utilizes optical chambers 190 further embodiments may omit the optical chamber in favor of other light sources or other constructs for passing energy from the at least one light sources 175 onto or through the at least one target with the entrapped reagents 210, 240. Similarly, the light energy may be directly incident and reflected from the surface of the at least one target 210, 240. Additionally, though the at least one sensor 170 is shown to be on the same side of the housing 100 as the at least one light source and receives indirect energy reflected back to it, the at least one sensor or detector 170 may also be reconfigured to sit opposite the at least one light source so as to receive light passing directly through an at least one target 200. The at least one sensor or detector 170 can be, but is certainly not limited to an at least one spectrophotometer or a photodetector. Non-limiting examples of a photodetector include an at least one of a CMOS chips, CCD chips, photodiodes, photoresistors, phototransistors, phototubes and the like.

A printed circuit board 160 is provided with a controller 165 coupled thereto. The controller 165 communicates and controls the at least one light source 175, the at least sensors 170, and manages the power supplied to each as well as receives data from the at least one sensor 170. The controller 165 also processes the data from the at least one sensor 170.

The operation of the exemplary embodiment shown in FIGS. 1-3 can be summarized by starting at the at least one light source 175 projecting light energy into a water flow, as evidenced by the directional arrows. The at least one light source 175 may also shine and pass through a diffuser or filter prior to being reflected. Water flows in pipe 105 into and past the reflector chamber or portion 155 of the housing 100. The at least one light source produces a specific energy signature as the light energy is reflected from the at least one light source 175 incident into and upon the walls that form the curved reflecting portion 155 of the housing 100 and this energy is reflected back toward the at least one targets 210, 240. The resulting light energy interacts with the immobilized reagent entrapped in the at least one target 210, 240. The interaction of the light energy and the reaction of the reagent and the reactant in the water on the surface of the at least one target 210,240 produces optically detectable changes in the energy profile from light sources. This energy passes through the target 210, 240 and into the light chamber 190 where it is detected by the at least one sensor 170.

In this exemplary embodiment, the light chamber 190 is coupled with a sensor 170 so as to additionally prevent light from other reagent pads reaching the specific sensor. The light chamber 190 can also hold a filter for the sensor in further exemplary embodiments. The light chamber 190 can also be designed to diffuse the light energy further so that the sensor sees uniform intensity. This detection of energy by the sensor 170 is reported to the controller which correlates the measured change in the energy profile received by the at least one sensor 170 into a measurement of the desired variable, for example, but certainly not limited to, at least one of the pH, calcium ion concentration, free chlorine concentration, total chlorine concentration, cyanuric acid concentration, dissolved carbon dioxide concentration, dissolved oxygen concentration and the like.

FIG. 3 shows a further cross sectional view along the width of the housing of the exemplary embodiment of FIG. 1. As seen from this bottom oriented view the at least one target having an immobilized reagent is shown as multiple Sol-Gel constructed targets 200, 210, 220, 240 contained in a ring surrounding window 180. The relationship of window 180 relative to the at least one target 210, 240 is more clearly shown in the exemplary embodiment. The at least one light source 175 relative to the window 180 is also more easily seen, with the at least one Sol-Gel targets 200, 210, 220, 240, and the at least one light chambers 190 for each.

It should be noted that each of the indicated targets 200, 210, 220, 240 can have a different immobilized reagent. The different immobilized reagents may respond to the presence of such analytes or reactants as, but are certainly not limited to, free chlorine, total chlorine, hydronium ions, calcium ions, and the like as noted. In addition, any of the spaces for the Sol-Gel targets, for instance target 230, can be empty or have a clear target. The clear space or window 230 is used for calibration and detection of fouling, light source deterioration, water turbidity, or similar diagnostic features. The light passing through the clear space or window 230 is unchanged due to changes in the water chemistry and any turbidity, deterioration, or other variance caused by the water or malfunctions in the system would be easily detectible as part of a calibration diagnostic like that discussed in relation to FIG. 5A. The calibration would be a component of controller 160 and the software contained thereon. Again, as described in relation to FIG. 2 above, the at least one sensor 170, here a set of sensors for each target 210, 240, including the calibration target, detects light energy emitted by the at least one light source 175 back into the light chambers 190 and onto the at least one sensors or detectors 170. The light passes through and is changed by interactions with optically detectable interactions occurring on each of the targets, with the exception of the window or clear target 230, the interactions change the energy received by the sensors 170. The data received at the sensors 170 is then provided to the controller 165 and correlated to eventual readings reported in the user interface 140.

FIG. 4 shows a plan view of an exemplary embodiment of this invention. The plan view emphasizes the operational aspects of the invention. A controller 600 is provided with a power management component 601 and a communications component 603. The controller 600 controls a light source driver 620 which is communication with an at least one light source 621-626. These light sources 621-626 in the exemplary embodiment can emit different bands of wavelength and the light source driver 620 is able to independently drive the light sources 621-626 at desired intensity. The light sources emit light that is incident on an at least one target 610, 611 having an immobilized reagent that reacts with reactants in the flowing water as an indicator of properties of the water. This interaction is measured as a change in the spectrographic absorption at the at least one target 610, 611 and this change is detected by an at least one sensor 606, 607. Similarly, a calibration or reference window 615 is provided with a target that is clear or has no doping of an immobilized reagent or is otherwise non-reactive with the reactant 615 which likewise receives energy from the at least one light source 621-626 but does not have an interaction occurring that changes the energy in a manner like the at least one target with the immobilized reagent. The reference window 615 acts as a calibration target and passes the known light profile emanating from the at least one light source 621-626. Variations in this profile indicate calibration issues which may result from conditions in the sample, for instance but not limited to turbidity, conditions in the at least one light source, for instance but not limited to light source degradation or malfunction, or when compared to other sensor results may be able to provide identification of sensor malfunctions. The at least one target component 610, 611, 615 can be removed from the system and replaced or changed to suit the environment and use of the sensor system.

Though multiple light sources are provided, a single light source may also be provided. In this exemplary embodiment, the multiple light sources 621-626 are individually addressable sources that are driven by the light source driver 620 in communication with the controller 600. The individual lights 621-626 in this case can provide light of specific narrow bands of wavelengths based on instructions from the light driver 620. The use of narrow or broad band light sources is fully contemplated and these can be used in conjunction with one another, alone or in any myriad of combinations, to provide the requisite incident energy for spectrometric analysis of the resulting light incident on the analyte and reactant interaction. These will in turn create a profile of intensity over a wavelength band, an example of which is shown in FIG. 12, having specific levels of intensity across the profile for individual reactants with the reagents. The result is that when the reagent-reactant interaction occurs, a change in this pattern will occur in one or more of these sources and be detectable as an indicator of the reactant in the water. This is correlated to a specific target variable being detected by the sensors 606-608 and the controller 600.

In addition the controller 600 is in communication with at least one temperature sensor, thermistor, thermopile, infrared sensing, thermocouple and the like 640, at least one salinity sensor 642, and at least one displacement based flow sensor, differential pressure sensor, inductive flow sensor, coriolis flow sensor, ultrasonic flow sensor, calorimetric flow sensor and the like. These additional sensors 642, 640 located in a pipe 105 having water 633 flowing within it and through it in the direction of the arrows. The pipe 105 has a first wall 631 a second wall 632 the sensor arrangement has immobilized reagents 610, 611 contained therein. The controller 600 is in further communication with a signal conditioning circuit 605 which feeds signals from the at least one detector 606, 607, 608 to the controller 600. The controller analyzes the variables relating to the various reagent-reactant reactions being detected by the at least one detector 606-608 and communicates the results through the communication component 603. This can be communicated to a user interface, as seen for example in the exemplary embodiment of FIG. 1, or off from this controller 600 via a wired or wireless connection to a further master controller, for instance a pool management panel or to a wireless hand held device coupled via a local area network or home network.

FIG. 5A shows a flow chart for a method of calibrating an exemplary embodiment of the instant invention. The process described is for use with the controller 600 or similar controlling devices which may reside elsewhere in the water treatment system and thereby act as a master controller over the sensor system. The calibration process is used by the controller to calibrate the sensors before operations begin, typically in a factory setting. However, similar steps may be incorporated in on-board calibration for use during operation as well. The calibration process 800 has a first step whereby each sensor output is registered in dark conditions and the resulting values obtained are stored as calibration data in step 810. The at least one light sources are then, independently or simultaneously, enabled and light is emitted into the system in step 820. The sensor output under this illumination condition is further stored as calibration data in the controller in this step. In step 830, the structure having surfaces with immobilized reagents is deployed. In addition to the surface targets with the immobilized reagents a calibration slot may be left as a reference, one without reagents. This step can be run during manufacture as well as in the field.

In step 840 the at least one light sources are again enabled, independently or simultaneously, enabled and light is emitted into the system. Again the output is stored as calibration data. A calibration solution having known properties and reactant levels is then admitted into the sensor system and the physical parameters are controlled as a known variable in step 850. The system is engaged and the data collected from the sensors for the known variables, as previously noted. Similarly, other variables, such as temperature, flow rate, turbidity, and the like are kept constant, at a known level. The outputs are measured and calibration data is stored in this step 850.

Again the at least one light source is then, independently or simultaneously, enabled and light is emitted into the system in step 860 while the system has the calibration solution in contact with the immobilized reagents. Again the output is saved as calibration data. In calibration step 870, the relationships between the measured interactions of the reagent with reactants and the resulting sensed light conditions are measured and stored. This is done through correlation of the measured outputs for all the calibration steps and for measured values for the resulting expected or known physical variable being measured by the reactant.

Finally, a look up table and equations for correlation of the measured interactions that are used during the normal measurement process by the device is completed in step 880. The resulting table is stored on the controller. Further calibration processes having similar steps may be performed to adjust a pre-loaded look up table during an installation or construction of the system. Similarly, after installation in a water treatment system, the calibration routine may be run again in total or in part to re-calibrate the system. As noted, some or all of these steps may be performed in the field, for instance to prepare for testing or during maintenance of an apparatus of the invention.

FIG. 5B shows a flow chart of an exemplary measurement process for use in the instant invention. The process described is for use with the controller 160 or 600 for the sensor system 10. Step 910 of the exemplary measurement process of FIG. 5B enables the light sources, independently or simultaneously, so that the sensors system can measure output of each sensor with reagent under the illumination conditions. The data obtained at the sensors is stored for the specific sensor.

In step 920, the light sources are again enabled, independently or simultaneously, and the results at the clear or reference window is measured and stored. In step 930 the relationships between absorption by the sensors with reagents and the reference window is calculated and the values of the calculations are stored for an unknown concentration of the target reactant. The data collected in steps 910-930 are then used in a look up table to correlate the reference measurement to a correlation measurement of a target component which is then processed to calculate a concentration of a reactant or target compound in step 940.

In step 950, a measurement of environmental conditions is then made, such as but certainly not limited to temperature, turbidity, flow speed, and similar measurements. The data so calculated in the look up table and the measurements of other variables in steps 920-950 are used to calculate the concentration a target component in the sample in step 960. The calculated and reported data is then saved with identifying variables, e.g. time, date, measurement conditions, and similar conditions, in step 970. The resulting output is an indicative sample and calculation of the intended variable to be measured. In step 980, the results that were stored are output to the user interface and display and other components if so configured. This reporting can also be done via wire or wirelessly or over a network.

FIG. 6 shows a further embodiment of the instant invention with a clamp on fixture that puts the instant invention in line with the water treatment system. The exemplary embodiment of the instant invention in FIG. 6 is similar to that shown in FIGS. 1-3, having a housing 300 with a curved reflector portion 395 and having a top 330 with a coupling member (not shown) and a grip member 335 to allow for coupling the top 330 to the housing 300. A power source 338 is coupled to a wire access port 337 to power the exemplary embodiment of FIG. 6. A user interface 340 is provided with an at least one user input 345 and a graphical display 350. A top collar portion is generally shown as 310, having an upper portion of the top collar portion 310 coupled to the housing 300 and the pipe 105. A lower collar portion 320 couples with the lower portion of the top collar portion 310, here using for example an at least one screw 326 to pull the top collar portion 310 and lower collar portion 320 together around the pipe 105 to drive a diverter portion into the water flow, as better shown in FIG. 7.

FIG. 7 shows a cross-sectional view of the exemplary embodiment of FIG. 6. The internal elements are more easily shown in this view. As noted, the housing 300 has a curved reflector portion 395 with a further curved reflector element 396 therein. As shown in the figure, the reflector element portion 396 is spaced slightly apart from the curved housing section 395. Above the reflector element 396, coupled to the housing top 330 are an at least one light source 375, a controller 365, a window 380, an at least one sensor or detector 370, an at least one light chamber 390 and an at least one immobilized reagent target or pad 400 similar to those components in the exemplary embodiment of FIG. 1.

The admission of the water into the reflector portion 395 of the exemplary embodiment is accomplished in a different fashion, as the upper and lower collar portions 310, 320 work to push a water inlet 316 and a water outlet 317 through and into the pipe 105 of the water treatment system. This may be done mechanically, in one non-limiting example, through the coupling of the upper and lower portions 310, 320 with the water inlet 316 and the water outlet 317 facing in opposed directions. The water inlet 316 is faced such that the opening 318 is opposed to the flow of water within the pipe 105 as indicated by the arrow. The water flow in the direction shown in 319 provides an overall pressure from the dynamic pressure of the flow at inlet opening 318 which is greater than the pressure at outlet 317 which only has static pressure. As a result, the water is forced through the tube 316 and into the chamber 395. A displacement flow sensor 363 is provided to measure the inflow of water. The water enters through the water inlet 316 and fills up and over the edge of the reflector portion 395 filling it like a bowl. A mount for the reflector portion 321 separates the water inlet 316 from the water outlet 317. The outflow of water exits the sensor system 10 through the water outlet 317.

Thus the water inlet 316 acts as a scoop drawing water into this exemplary embodiment of the instant invention. Other structures may be used to similarly redirect a sample of water from the pipe 105 of the water system to the sensor system 10 of the exemplary embodiment without departing from the spirit of the instant invention. In this configuration the water inlet 316 has a dynamic pressure from the movement of the water and feeds sample water into the reflector chamber 395 to be exposed to the at least one light source 375 so that it will illuminate the at least one immobilized reagent target 400 and thereby provide a reflected, modified light energy to pass into the at least one light chamber 390 and reach the at least one sensor 370.

The sensors 370 in the sensor system 10 measure the changes in the light passing through the at least one immobilized reagent pad 400. The controller 365 relates the measurements at the at least one sensor 370 to a value for a desired variable and displays same through the graphical user interface 350. The particular values displayed can be changed by the at least one user inputs 345 to cycle through the desired target variables and display same. The electrical connection 338 can also include a communication line (not shown) or a wireless communication link (not shown) in the controller 365 to communicate the sensed variables out to a master systems controller for the water treatment system. Thus, either through its controller 365 or through a master systems controller, the sensed variable can be used to adjust other components of the water treatment system.

FIG. 8 shows a sensing component of a still further exemplary embodiment of the instant invention. FIG. 8 shows a housing 705 with an at least one pump 720, 728 coupled to the housing and further diverter elements 733, 734 as shown in FIG. 9. An at least one lid 710 is coupled to the housing 705 by a pair of hinges 716 having an opening 714. An optical test chamber with one or more surfaces with an immobilized reagent 740 is shown. The optical test chamber again contains an at least one source of light which is incident on the one or more surfaces with a permanently immobilized reagent whereby the permanently immobilized reagent interacts with a reactant in the water sample brought to the chamber by diverter elements 733, 734. The reaction creates an interaction with the energy emitted by the at least one light source in the test chamber 740 and this is read by an at least one sensor and the data is transmitted to the controller 754. An at least one parameter or variable, as noted above, is measured by the optical test chamber 740.

An electronics section 760 including the controller, communication, power, light source control, and software with algorithms related to the measurements is provided within the housing 705. A power and communications coupling 730 is provided to couple the electronics section 760 to communications and electrical power sources (not shown). The housing 705 and lid 710 act to seal the electronics section 760 and test chamber 740 from the elements. A user interface 750 is provided with user inputs 752 which protrude from the lid 710 through cutout 714. The cut out 714 is further sealed against the elements with lid 710 is closed. The controller 754 receives input from the optical test chamber 740 representing the measurements of the sensors detecting variations in the light after interacting with the at least one surface having the permanently immobilized reagent embedded thereon. The controller in the electronics section 754 correlates this data with a resulting output for a specific variable in a method, for instance but certainly not limited to, the exemplary method of FIG. 5B for measurement. The output is displayed on the user interface 750. Based on this output, adjustments of the water management system can be input and additional chemicals may be pumped via pumps 722, 728 into the water treatment system.

The housing 705 can be mounted remotely from the sensing system 10. Lines 733, 734 coupling the water in the water treatment system to the test chamber 740 are provided and extend to the coupling system shown in FIG. 9. To facilitate mounting on a wall or other structure, a series of holes 706 are provided around the housing 705. The system operates on the similar core principals as that of the previously disclosed embodiments, the embodiment of FIGS. 8 and 9 simply makes it easier to identify and retrofit a version of the instant invention. It also provides additional mechanisms to correct the status of the water being monitored. The sensor system may instigate action by other elements of the water treatment system, one example being, but certainly not limited to, determining pH in a pool or spas.

FIG. 9 shows an exemplary embodiment of a diversion component of the still further exemplary embodiment of FIG. 8. One or more of these structures is provided for use with the controller of FIG. 8. The collar has an upper and lower member 769, 770 respectively, which are coupled by a hinge and a screw 768. The tube 764 feeds input 733 from FIG. 8 and allows a sample to pass to the optical test tubes 733, 734 from the optical test chamber 740 are each dropped to one of these couplings. The tubes 733, 734 couple to a tube fitting 761. This allows for water to be directed from a pipe 765 in the water treatment system to the optical test chamber 740 from a coupling at a higher pressure point in the system. The outlet 732 is similarly coupled to a lower pressure location on the pipe 765 for return to the system.

FIG. 10 shows a plan view of an exemplary embodiment of the instant invention deployed in a water treatment system servicing a pool and/or spa. Though a pool and/or spa is shown, further recreational uses can include but are certainly not limited to water parks, public pools, public ornamental water displays, private ornamental water displays, fountains, and the like. FIG. 10 shows the water treatment system, including a pump 530, a filter 540, a heater 550 and a dispenser with a CO₂ tank 570 with a master controller 504. The pool 510 is serviced by the system, with water 515 flowing there through. Relevant parameters measuring the quality of the water are taken by the sensor system in the immobilized reagent chemical sensor 500. The immobilized reagent chemical sensor allows for more accurate, cheaper, more easily maintained chemical analysis of desired structures using the previously described light sources with an optical detection setup. It can also report to the master controller 504 its variables and the master controller 504 can then adjust all the other components 530, 540, 550, 560, 570, 575 of the pool system. Alternately, the control of the sensor system may act to control and adjust the system.

FIG. 11 shows a plan view of an exemplary embodiment of the instant invention deployed in a water treatment system for a fish farm operation.

FIG. 11 has a water tank with fish 511. Various fish 517 are within the container. Although a fish farm tank is shown, further industrial uses contemplated for the instant invention include, but are certainly not limited to boilers, HVAC, water conditioning systems, industrial processing systems, food processing, industrial cleaning systems, potable water systems, waste water systems, environmental monitoring systems, agricultural systems, aquaculture systems, testing labs. A controller 504 manages the system and circulates water 516. The water is pumped into a loop having a surface aerator 580, a pump 530, a filter 540, and a bio filter 545, a chemical sensing package having one or more immobilized reagent chemical sensor 500 which can be for instance, but is certainly not limited to one of the exemplary embodiments of FIG. 1-9, a biomass container 590 a nutrient dispense 560 and a sensor array 525 that can be looking at further parameters such as temperature, pressure, ambient light conditions and the like. An oxygen tank with activation controls is also provided with a water input 575. Water flows within the water treatment system which requires additional filtration before re-admission. The fish 517 consume nutrients and thus have need of such output, here accomplished by nutrient dispenser 560. Additionally waste materials produced by the fish 517 are pumped from the water controlled by the controller unit 504. An air pump 587 further improves aeration in the water tank 511. The same type of immobilized reagent in a sensor is utilized regarding the exemplary embodiment of FIG. 11. Any of the exemplary embodiments of FIGS. 1-9 may be utilized in conjunction with the embodiments of FIGS. 10 and 11.

FIG. 12 is a diagram of a spectral profile of multiple light sources in an exemplary embodiment providing a controllable wavelength selection capability. The figure shows an optical profile, however this profile involves energy at specific wavelengths for multiple independently controlled sources. The use of narrow or broad band light sources is fully contemplated and these can be used in conjunction with one another, alone or in any myriad of combinations, to provide the requisite incident energy for spectrometric analysis of the resulting light incident on the analyte and reactant interaction. In the profile example shown, several intensity “peaks” show where specific, narrow band light sources are emitting energy 1010, 1020, 1030 in bands around specific wavelengths. These in turn are at specified wavelengths with specific dispersal across the profile. The result is that when the analyte-reactant interaction occurs, a change in this pattern will occur in one or more of these sources and be detectable as an indicator of the reactant in the solution. This will again be correlated to a specific target variable being detected by the sensor.

The embodiments and examples discussed herein are non-limiting examples. The invention is described in detail with respect to preferred embodiments, and it will now be apparent from the foregoing to those skilled in the art that changes and modifications may be made without departing from the invention in its broader aspects, and the invention, therefore, as defined in the claims is intended to cover all such changes and modifications as fall within the true spirit of the invention.

What is claimed is:

1. A sensor system in a water treatment system, comprising:
a housing;
a controller;
an at least one light source;
an at least one sensor; and
an at least one target having an at least one immobilized reagent with the at least one light source emitting light energy into the housing that is incident upon the at least one target with the immobilized reagent and the immobilized reagent being in contact with a sample of water from the water treatment system, wherein the at least one target having the immobilized reagent interacts with a reactant in the water such that the interaction changes the state of the reagent and when energy from the at least one light source is incident on the at least one target with the immobilized reagent the energy from the at least one target having the at least one immobilized reagent shows a change detectable by the at least one sensor such that the changed energy is detectable by and collected at the sensor and data on the energy is communicated to the controller, the data is then correlated as a representation of a desired variable to be measured for the water in the water treatment system.

2. The sensor system of claim 1, wherein the at least one target further comprises multiple targets with immobilized reagents.

3. The sensor system of claim 1, further comprising multiple reagents embedded in the at least one target.

4. The sensor system of claim 3, wherein the multiple reagents are on multiple targets.

5. The sensor system of claim 1, wherein the immobilized reagent is at least one of an at least one organic or inorganic dyes.

6. The sensor system of claim 5, wherein the at least one organic or inorganic dye is at least one of bromocresol green, cresol red, bromothymol blue, bromopyrogallol red, phenol red, orthotolidine, N-N, diphenyl-p-phenylenediamine, and melamine.

7. The sensor system of claim 1, wherein the immobilized reagent that is at least one of an at least one enzyme.

8. The sensor system of claim 7, wherein the at least one enzyme is at least one of Aequorin, Chloramine, and Glucose Oxidase.

10. The sensor of claim 1, wherein the variable is measured by a concentration of the reactant and the reactant is an at least one dissolved reactant in the water.

11. The sensor system of claim 10, wherein the dissolved reactant is an at least one ion.

12. The sensor system of claim 11, wherein the at least one ion is at least one of an at least one hydronium, chlorine, calcium, iron, sodium, lead bromine, magnesium, and copper ion.

13. The sensor system of claim 5, wherein the dissolved reactant is an at least one compound.

14. The sensor system of claim 13, wherein the at least one compound is an at least one of an at least one oxygen, carbon-dioxide, cyanuric acid, chlorine, and glucose compound.

15. The sensor system of claim 13, wherein the variable is measured by a concentration of at least one of a flora and fauna.

16. The sensor system of claim 15, wherein the at least one flora and fauna is an at least one algae and bacteria.

17. The sensor system of claim 1, further comprising a reflector portion or chamber.

18. The sensor system of claim 1, further comprising and at least one additional sensor sensing an at least one of the flow rate, temperature, humidity, ambient light conditions, free chlorine, and salinity of the water.

19. The sensor system of claim 1, wherein the controller is within the housing in an electronic section also housing the at least one light source and the at least one sensor.

20. The sensor system of claim 1, further comprising an at least one window separating the at least one light source from a flow of water within the housing, wherein the targets are spaced around the window and the sensors are located proximate to the at least one target.

21. The sensor system of claim 1, further comprising a reflective portion of the housing whereby light emitted by the at least one light source and is emitted through the window and is reflected back within the reflective portion back toward the at least one target and passes through the target into a light chamber which aids in collecting and focusing the reflected light onto the at least one sensor above the target.

22. The sensor system of claim 1, wherein the controller is outside of the housing.

23. The sensor system of claim 1, further comprising a user interface.

24. The sensor system of claim 1, wherein the controller is located with the user interface.

25. The sensor system of claim 1, wherein the controller is located on the housing and includes a communication subsystem for wired or wireless communication with a graphical user interface.

26. The sensor system of claim 1, wherein the housing is in line with a plumbed water line in the water treatment system.

27. The sensor system of claim 1, wherein the at least one sensor is a photodetector.

28. The sensor system of claim 27, wherein the at least one photodetector includes an at least one spectrometer, CMOS chip, CCD chip, photodiodes, photoresistors, phototransistors, and phototubes.

29. The sensor system of claim 1, where the targets are directly in the line of flow.

30. The sensor system of claim 1, where the water flow is redirected from the main line of water flow to the targets and then back to the main line of water flow.

31. The sensor system of claim 1, wherein the housing containing the target is in the flow of water.

32. The sensor system of claim 1, wherein the housing containing the target is in a body of water being serviced by the water treatment system.

33. The sensor system of claim 1, wherein the housing is coupled via a pressure differential to divert a sample of the water in the water treatment system to the sensor system.

34. The sensor system of claim 1, wherein the housing is coupled to the a pipe in the water treatment system through an upper and lower collar portion with an inlet and an outlet path extending from the housing into the pipe to redirect water into the housing.

35. The sensor system of claim 1, wherein the sensor system includes an at least one additional sensor additionally measuring an at least one or more of temperature, humidity, ambient light conditions, free chlorine, flow displacement, and differential pressure.

36. The sensor system of claim 1, further comprising an at least one calibration target or blank, where light incident on the at least one immobilized reagent target is also incident upon the calibration target or blank without an interaction and variations in the profile of the energy emitted from the at least one light source is detected by the at least one sensor, whereby any variations in the received profile are used to adjust the sensors and correct the data for the light received that is incident on the at least one immobilized reagent target.

37. The sensor system of claim 36, wherein variation in the profile of the energy emitted and received by the at least one sensor at the at least one calibration target or blank are stored by the controller.

38. The sensor system of claim 1, wherein stored data on variations in the profile of the energy emitted is reviewed by the controller and the controller can categorize and thereby detect profiles for fouling of the water treatment system flow of water, errors from the at least one light source, errors from one or more of the at least one sensors, and the stored data can be compared against calibration data stored during manufacture of the sensor system.

39. The sensor system of claim 1, wherein the data correlated as a representation of a desired variable to be measured for the water in the water treatment system is communicated through a user interface.

40. The sensor system of claim 39, wherein the user interface is on the housing.

41. The sensor system of claim 39, wherein the user interface is wirelessly coupled to the controller.

42. The sensor system of claim 41, wherein the user interface is a mobile computing device.

43. The sensor system of claim 39, wherein the user interface is coupled via a wired coupling to a user interface outside the housing.

44. The sensor system of claim 1, wherein the at least one light source is an at least one of an at least one incandescent lights, halogen lights, white (phosphorous coated) lights, LEDs, and HID.

45. The sensor system of claim 1, wherein the at least one target with an immobilized reagent is comprised of material formed by a Sol-Gel process

46. The sensor system of claim 45, wherein the matrix is formed using a metal alkoxide or a metal alkyloxide precursor compound in the Sol-Gel process.

47. The sensor system of claim 46, wherein the precursor compound is one or more of Tetraethoxy silane (TEOS), Tetramethoxy silane (TMOS), and Methyltrimethoxy silane (MTMOS).

48. The sensor system of claim 46, wherein the Sol-Gel formed material is at least one of an at least one thin film, bulk material and dense ceramic.

49. The sensor system of claim 36, wherein the optical profiles detected by the sensors are stored on the controller along with calibration profiles as historical data.

50. An at least one sensor system coupled to a pool, spa, or water feature water treatment system having water flowing within the water treatment system, the sensor system comprising:
an at least one housing
an electronic section containing at least one light source, at least one controller;
at least one sensor;
an at least one immobilized reagent target;
an at least one sensor sensing energy incident on or through the at least one immobilized reagent target, wherein the at least one light emits an energy with specific known optical profile which is then incident on the at least one immobilized reagent target which is in contact with the water from the water treatment system and the immobilized reagent interacts with the water sample to produce a reaction in or on the at least one immobilized reagent target which changes the energy profile on the at least one immobilized reagent target, the changes are then detected by the at least one sensor sensing energy incident on or through the at least one immobilized reagent target.

51. The sensor system of claim 50, further comprising a calibration target or blank, where light incident on the at least one immobilized reagent target is also incident upon the calibration target or blank without an interaction and variations in the profile of the energy emitted from the at least one light source, whereby any variations in the received profile are used to adjust the sensors and correct the data for the light received that is incident on the at least one immobilized reagent target.

52. The sensor system of claim 50, housing is provided as a component of the existing water treatment system.

53. The sensor system of claim 50, the housing being placed in-line with a portion of the piping of the water treatment system

54. The sensor system of claim 50, wherein the at least one light source is at least one of an at least one ultraviolet, infrared, and visible light.

55. The sensor system of claim 50, further comprising an at least one display, user interface, and user input.

56. The sensor system of claim 552, wherein the display and user inputs are digital and incorporated into a touch pad device.

57. The sensor system of claim 55, wherein the display and user interface are coupled wirelessly or coupled with a wired connection to the controller or to a master controller.

58. The sensor system of claim 50, wherein the sensor system controls one or more further components of the water treatment system.

59. The sensor system of claim 58, wherein the one or more further components is at least one of an at least one chlorine generator, acid dispenser, water treatment filter, and water pump.

60. The sensor system of claim 50, wherein the housing is remote from the water system and includes an at least one diverter to sample water from a portion of the water treatment system.

61. The sensor system of claim 50, wherein the housing has an upper and lower collar portion and is coupled through a pipe within the water system by the collar portions.

62. The sensor system of claim 50, wherein the immobilized reagent that is at least one of an at least one organic or inorganic dyes.

63. The sensor system of claim 58, wherein the at least one organic or inorganic dye is at least one of bromocresol green, cresol red, bromothymol blue, bromopyrogallol red, phenol red, orthotolidine, N-N, diphenyl-p-phenylenediamine, and melamine.

64. The sensor of claim 50, wherein the variable is measured by a concentration of the reactant and the reactant is an at least one dissolved reactant in the water.

65. A method of sensing a reactant in a body of water, comprising the steps of:
directing a sample of water from a body of water into a housing having an at least one detection targets with an immobilized reagent thereon;
directing an at least one light source incident upon the at least one detection targets having immobilized reagents thereon;
emitting energy from the at least one light source incident upon the at least one detection targets having immobilized reagents thereon such that the energy changes with any interaction the immobilized reagents have with the sample;
detecting a change in the energy incident upon the at least one detection targets having immobilized reagents caused by the interaction of the immobilized reagents with the sample of water; and
reporting the results of the detection step.
